(19) United States
(12) Patent Application Publication
(10) Pub. No.: US 2001/0009774 A1
(43) Pub. Date: Jul. 26, 2001

(54) INTERFACE SENSING MEMBRANE IN BIOELECTRONIC DEVICE AND METHOD FOR FORMING THE SAME

(76) Inventors: MIN CHOL SHIN, SEOUL (KR); SEUNG RYEOL KIM, SEOUL (KR); TAE HAN KIM, SEQUL (KR); KANG SHIN LEE, KYUNGKI-DO (KR); WON YONG LEE, KYUNGKI-DO (KR); JE KYUN PARK, SEOUL (KR)

Correspondence Address: THE LAW OFFICES OF, FLESHNER & KIM, POST OFFICE BOX 221200, CHANTILLY, VA, 201531200

(21) Appl. No.: 09/221,984

(22) Filed: Dec. 29, 1998

(30) Foreign Application Priority Data
Dec. 30, 1997 (KR) 79064/1997

(51) Int. Cl. G01N 33/543

(52) U.S. Cl. 436/518

(57) ABSTRACT

Interface sensing membrane in a bioelectronic device and a method for forming the same, the device including a molecular adhesive layer chemically adsorbed to a surface of a solid state element, a three dimensional microstructured layer of a hydrophilic polymer connected to the molecular adhesive layer in a covalent bond, and a bioelement immobilized in the three dimensional microstructured layer by covalent adhesive, whereby causing a stable biomolecular interaction.

### BACKGROUND OF THE INVENTION

1. Field of the Invention

The present invention relates to an interface sensing membrane in a bioelectronic device and a method for forming the same, which is provided with a three dimensional microstructure for immobilizing a bioelement at a surface of a solid state element, for interfacing the surface of the solid state element and an outside of the bioelectronic device, i.e., for causing a biomolecular specific interaction within the three dimensional microstructure.

2. Background of the Related Art

The bioelectronic device, an electronic device to which a bioelement is introduced, is used for diagnosis and analysis, and as a part of an entire circuitry system and the like in various industrial fields, such as medical measurements, food analysis, or environmental measurements. As a typical bioelectronic device, there is a protein-based memory using a bacteriorhodopsin, a DNA chip, or biosensor. A biosensor is a device or an apparatus which employs a molecular recognition function of various biomaterials for measuring chemicals. The molecular recognition function is a function, in which a biocatalyst, such as an enzyme, a microorganism, or a cell makes a specific response to a particular material. C. R. Lowe defined the biosensor, a combination of a bioelement and a physicochemical element, i.e., a solid state element, as a spatial integration of a transducer and a biomaterial(BIOSENSOR, 1985, 1:3-16). The biosensor is in general classified according to kinds of the bioelements which have a molecular recognition function and the solid state elements as physicochemical devices(transducers). For example, the biosensors may be classified as follows according to the bioelement used therein; a microbial sensor of an immobilized microorganism membrane, an immunosensor of an immobilized anti-body, an organelle sensor of an immobilized organelle, a tissue sensor of an animal or botanical tissue. Other than the molecular recognition function, the bioelectronic device, including the biosensor, requires a part for detecting a biological response and converting it to an electrical signal which is easy to be processed. In the present invention, the part for converting to an electrical signal is called as the solid state element, inclusive of the transducer and the like. In the transducers converting an enzyme reaction to an electrical signal, there are an oxygen electrode, hydrogen peroxide electrode, pH-ISFET(ion selective field effect transistor), and the like.

In the bioelectronic device, the question that what kind of bioelement is to be combined with what kind of solid state element depends on what kind of materials are mixed in a solution to be measured other than an object material and how the biosensor will be used, and the like. Thus, the bioelectronic device is based on a principle in which a chemical(s) consumed or produced by a reaction of the bioelement is detected with an electrode in the solid state element, and converted into an electrical signal from which an object chemical is measured. Because most of the bioelements are water soluble, the bioelements are required to be immobilized in . . . a polymer membrane for making the bioelements insoluble in water. The immobilization of the bioelement leads to maximize a measured signal, minimize an interference, and allow a multiple use of the bioelectronic device. There are many known arts for immobilizing the bioelement at a surface of solid state element, such as transducer. As traditional immobilization methods, there are carrier binding method, cross-linkage method, entrapping method, and adsorbing method.

The immobilization of the bioelement on a surface of a solid state element, such as a transducer, is a rather sophisticated method in which the bioelement is brought to a particular micron location at an interface between an inorganic material and an organic material. If the bioelement is happened to be brought into a direct contact with a solid state element of a metal or an inorganic material, there are problems caused in that the molecular recognition function of the bioelement is lost, losing the function as a bioelectronic device, and a nonspecific binding is caused by a non-reversible adsorption of an external biomaterial which is brought into contact with the interface of a surface of the device. EP 254575 introduced a solvent casting technique in which a polymer, such as cellulose nitrate, is coated on a surface of a solid state element, for adsorbing, and immobilizing a bioelement on the surface of the solid state element, which however can not solve the problem of the nonspecific binding caused by other biomaterial in a sample. U.S. Pat. No. 5,332,479 discloses formation of a thick film on an electrode surface and adsorption, and immobilization of a polymer, an enzyme, or an electroactive substance by screen printing. S. Nakamoto et al. employed an ink jet dispensing for immobilize polymers, and the like(Sens. Actuators, 1988, 12: 165). And, Umana and Waller describes a method for capturing the bioelements into a conducting polymer obtained by electrochemical polymerization(Anal. Chem. 1986, 58:2979). However, though a precise adjustment of the immobilization location on the surface of the solid state element is possible, the foregoing methods have a problem in that there is a limitation in that the immobilization of the bioelement can not be maintained for a prolonged time period because the bioelement is immobilized by noncovalent bonding, which may cause a leak of the bioelement. U.S. Pat. No. 4,562,157 discloses a method in which a specific protein is immobilized on a silicon surface by employing a photoresist and lift-off technique using UV irradiation. However, this method also has inconvenience of additional processing of a denaturant after the bioelement is immobilized for solving the problem caused by the nonspecific binding. U.S. Pat. No. 5,629,213 suggests a method in which a polyanionic material is attached to a gold film which is used as an SPR biosensor, a polycationic material is process thereon, on which surface a bioelement is immobilized. However, since the opposite polyionic materials on the sensor surface are maintained by electrostatic attraction, a stability of the polyionic materials on which the bioelement is immobilized can be affected by a pH and an ionic strength in the sample. U.S. Pat. No. 5,436,161 describes an immobilization of a bioelement on a matrix coating on a gold thin film in an SPR biosensor. The matrix coating is mainly formed of swellable polymer, for example, polysaccharide. Therefore, a separate activation step is required for immobilizing the bioelement on the inert matrix coating.

As discussed, the related art bioelement has problems in immobilizing the bioelement at a solid state element in that the bioelement is inactivated during an immobilization reaction, an activity of the bioelement is not stably maintained on the surface after the immobilization, or a non-specific binding of the other external biomaterials on the surface of the solid state element is caused.

### SUMMARY OF THE INVENTION

Accordingly, the present invention is directed to an interface sensing membrane in a bioelectronic device and a method for forming the same that substantially obviates one or more of the problems due to limitations and disadvantages of the related art.

An object of the present invention is to provide an interface sensing membrane in a bioelectronic device and a method for forming the same, which can prevent the bioelement from being inactivated during an immobilization reaction, an activity of the bioelement from being not stably maintained on the surface after the immobilization, and a non-specific binding of the other external biomaterials on the surface of the solid state element from being caused.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the interface sensing membrane in a bioelectronic device includes a molecular adhesive layer chemically adsorbed to a surface of a solid state element, a three dimensional microstructured layer of a hydrophilic polymer connected to the molecular adhesive layer in a covalent bond, and a bioelement immobilized in the three dimensional microstructured layer by covalent bonding.

The solid state element may be a gold thin film, a silver thin film, or silicon or glass slide.

The three dimensional microstructured layer may consist of molecules of polypeptides.

And, the molecular monolayer may consist of molecules which are heterobifunctional molecules containing a functional group for making a chemical adsorption reaction with the surface of the solid state element and a functional group for making a covalent bond with the three dimensional microstructured layer. The functional group for making a chemical adsorption reaction with the surface of the solid state element may be sulfur, or silane, and the functional group for making a covalent bond with the three dimensional microstructured layer may be carboxyl, aldehyde, amino, sulfhydryl, or hydrazide.

In other aspect of the present invention, there is provided a method for forming a molecular adhesive layer in a bioelectronic device including the step of causing the heterobifunctional reagent to make reaction with the solid state element.

The heterobifunctional reagent is preferably has the following chemical formula;

X-(CH₂)n-Y, where, X denotes the functional group causing a chemical adsorption with the solid state element and Y denotes the functional group making a covalent bond with a hydrophilic polymer.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention:

In the drawings:

FIG. 1 illustrates an interface sensing membrane in a bioelectronic device in accordance with a preferred embodiment of the present invention, schematically.

### DETAILED DESCRIPTION OF THE PREFERRED EMBODIMENT

The present invention provides an interface sensing membrane in a bioelectronic device, including a solid state element, molecular adhesive layer chemically adsorbed to a surface of the solid state element, a three dimensional microstructured layer of a hydrophilic polymer connected by a covalent bond with the molecular adhesive layer, and a bioelement immobilized by a covalent bond with the three dimensional microstructured layer. The solid state element may be a gold thin film, silver thin film, silicon, or glass slide. And, the three dimensional microstructured layer may consist of molecules of polypeptides, and the molecular adhesive layer may be a molecular monolayer and may consist of molecules each having a functional group which makes a chemical adsorption reaction with the surface of the solid state element and a functional group which makes a covalent bond with the three dimensional microstructured layer. The functional group which makes a chemical adsorption reaction with the surface of the solid state element is preferably a functional group containing sulfur or silane, and the functional group which makes a covalent bond with the three dimensional microstructured layer is preferably a functional group containing carboxyl, aldehyde, amino, sulfhydryl, or hydrazide. The three dimensional microstructured layer is preferably formed of a material or materials selected from polyglutamic acid, polyaspartic acid, polylysine, and polycystein.

The present invention also provides a method for forming the aforementioned molecular adhesive layer in a bioelectronic device by causing a heterobifunctional reagent to make reaction with a solid state element. Preferably, the heterobifunctional reagent has the following formulae.

X-(CH₂)n-Y,

Where, X denotes the functional group causing a chemical adsorption with the solid state element, and Y denotes the functional group making a covalent bond with a hydrophilic polymer. Preferably, X is the functional group containing sulfur or silane, and Y is the functional group containing carboxyl, aldehyde, amino, sulfhydryl, or hydrazide. X may be selected from —SH, —S— SH, —SiCl₃, —Si(OCH₃)₃, and Si(OCH₂CH₃)₃. In detail, the method for forming an interface sensing membrane in a bioelectronic device in accordance with a preferred embodiment of the present invention includes the steps of applying a solution containing a heterobifunctional reagent to a surface of a solid state element to form a molecular adhesive layer chemically adsorbed to the solid state element, making a hydrophilic polymer to cause a covalent bond with, and on the molecular adhesive layer by means of a coupling reagent to form a three dimensional microstructured layer, and making a bioelement to cause a covalent bond with the three dimensional microstructured layer. The steps for forming the molecular adhesive layer includes A) blowing in an inert gas while exposing the solid state element to the heterobifunctional reagent, and B) drying the solid state element in a nitrogen chamber, to form the molecular monolayer.

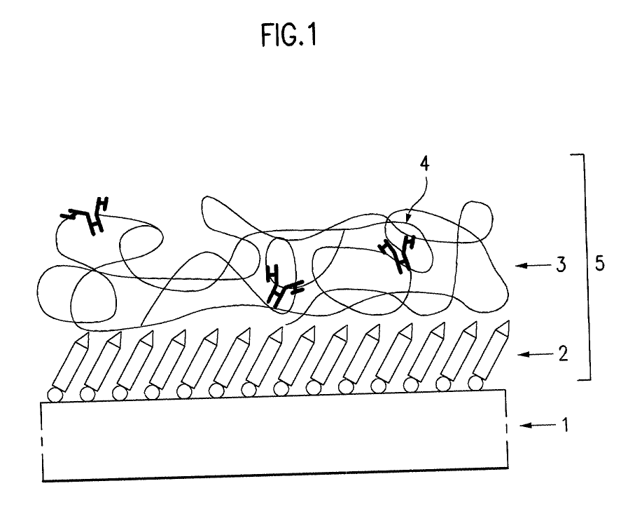

Reference will now be made in detail to the preferred embodiments of the present invention, examples of which are illustrated in the accompanying drawings. FIG. 1 illustrates an interface sensing membrane in a bioelectronic device in accordance with a preferred embodiment of the present invention, schematically.

Referring to FIG. 1, a molecular adhesive layer 2 is formed on a surface of a solid state element 1 by chemical adsorption. A three dimensional microstructured layer 3 of a hydrophilic polymer connected to the molecular adhesive layer 2 in a covalent bond is formed on the molecular adhesive layer 2, and a bioelement 4 is immobilized in the three dimensional microstructured layer 3, thereby completing formation of the interface sensing membrane 5. The solid state element 1 may be a noble metal film of gold, or silver, a silicon chip, or a glass substrate. The noble metal film of gold, or silver is a material for an electrode as well as a measuring instrument utilizing surface plasmon resonance. The silicon chip is used for fabrication of a bioelectronic device and as a substrate for a semiconductor or integrated circuit. The glass substrate is possible to make an association with an optical device. The molecular adhesive layer 2 is a molecular monolayer for serving as a connecting link for hydrophilic biopolymer to a surface of the solid state element, consisting of molecules each having a functional group which makes a chemical adsorption reaction with the surface of the solid state element and a functional group which makes a covalent bond with the three dimensional microstructured layer. The molecular adhesive layer 2 can be obtained by applying a heterobifunctional reagent of molecules each having different functional groups at opposite ends thereof to the solid state element. Molecular formula of the heterobifunctional reagent is as follows.

[X-(CH₂)n-Y],

That is, an X as a functional group for making a reaction with the surface of the solid state element and a Y as a functional group for adhesive with a hydrophilic polymer forming the three dimensional microstructured layer are respectively bonded to opposite ends of an inert carbon chain. Preferably, the functional group X may contain sulfur or silane, and the functional group Y may contain carboxyl, aldehyde, amino, sulfhydryl, or hydrazide. And, more preferably, —SH or —S—SH may be selected as a functional group X containing sulfur, and —SiCl₃, —Si(OCH₃)₃, or Si(OCH₂CH₃)₃ may be selected as a functional group X containing silane. The molecular adhesive layer 2 can be obtained when the functional group X in a molecules of the heterobifunctional reagent makes a thermodynamically spontaneous chemical adsorption reaction with the surface of the solid state element, to form a molecular monolayer on the surface of the solid state element. It is known that a molecule with a functional group containing sulfur forms a very close packed molecular monolayer by making a covalent bond with a surface of a gold or silver film through thiolate(R. G. Nuzzo et al. J. Am. Chem. Soc. 1983, 105:4481). And, it is known that a molecule with a functional group containing silane forms a molecular monolayer by making a covalent bond with the surface of the solid state element of silicon or glass at forming a membrane of two dimensional network of the silane molecules on the surface of the solid state element(K. M. Rusin et al. Biosens Bioelectron. 1992, 7:367). And, another functional group Y in the molecule of the heterobifunctional reagent is a functional group which can make a covalent bond with a hydrophilic biopolymer which forms the three dimensional microstructured layer. The functional group Y is preferably carboxyl, aldehyde, amino, sulfhydryl, or hydrazide. At a side opposite to the side of the functional group X in the molecular adhesive layer, which makes a covalent bond with the surface of the solid state element, the functional group Y makes a covalent bond with the hydrophilic biopolymer. Thus, the three dimensional microstructured layer 3 of a hydrophilic biopolymer connected to the surface of the solid state element through the molecular adhesive layer and, in turn, the hydrophilic biopolymer is connected to the bioelement in a covalent bond, eventually connecting the bioelement to the solid state element. Thus, the three dimensional microstructured layer 3, not only facilitates an easy immobilization of the bioelement to the surface of the solid state element, but also provides an environment which is susceptible to cause a biospecific interaction that can maintain a high activity of the bioelement even after the immobilization. The hydrophilic biopolymer is preferably a molecule of polypeptides, and, more preferably, may be selected from a group containing polyglutamic acid, polyaspartic acid, polylysine, and polycystein. There are functional groups, such as carboxyl, amino, or sulfhydryl in the biopolymer. The functional group acts as a portion that makes a covalent bond in immobilizing the bioelement to the three dimensional microstructured layer 3.

The last step of the method for forming an interface sensing membrane 5 in a bioelectronic device is a step for immobilizing the bioelement 4 in the three dimensional microstructured layer of a hydrophilic biopolymer. The bioelement employed in this case in general acts as a receptor in view of a relation of receptor-ligand. Pairs of receptor-actor applicable to the interface sensing membrane of the present invention are as follows; antbody-antigen, protein A and G-immunoglobulin G and M, enzyme-substrate, avidin-biotin, avidin-biotinylated biomolecule, DNA-DNA, DNA-RNA, lectin-polysaccharide chain, a general receptor present in a membrane or the like-a general ligand such as hormone.

### EMBODIMENT 1

The interface sensing membrane using avidin-biotin interaction

(1) Formation of a gold thin film

The gold thin film to be used as a substrate is formed as follows. A chrome or titanium film is deposited or sputtered on a clean glass slide to a thickness of 5-10 nm. This thin film enhances an adhesive strength of gold. A gold thin film is formed thereon by the same method to a thickness of40-200nm. Then, a diamond stylus is used to cut the substrate by 1×1 cm², which is used in the next step to form the interface sensing membrane.

(2) Formation of a molecular adhesive layer on the gold thin film

A process for forming the molecular adhesive layer on the gold thin film using a heterobifunctional reagent will be explained. A cystamine used as the heterobifunctional reagent is a disulfide having an amine functional group at both sides thereof. 1 mM of cystamine solution is prepared using deionized pure water with a resistance of 18MΩ after deaeration. After 10 ml of the heterobifunctional reagent solution is filled in a glass scintillation vial, a specimen of the gold thin film is placed therein. Inert gas, such as argon is blown into the vial, to expel air in a head space of the vial. Then, while being kept air tight, the vial is left standstill for more than 18 hours, to cause chemisorption reaction between the cystamine water solution and a surface of the gold thin film specimen to form a molecular monolayer on the surface. A surface of the molecular adhesive layer is repeatedly washed with ethanol and water, dried with nitrogen gas, and conserved in a nitrogen chamber.

(3) Formation of three dimensional microstructured layer on the molecular adhesive layer

The hydrophilic three dimensional micro structured layer can be formed on the molecular adhesive layer according to the following process. Poly-L-glutamatic acid(PGA) is used as the hydrophilic polymer, and N-ethyl-N′-(dimethylaminoprophyl) carboimide(EDC) and N-hydroxysuccinimide(NHS) are used as coupling reagent between the molecular adhesive layer and the hydrophilic polymer. A triethanolamine(TEA) buffer solution(0.05M, pH 8.0) containing 1 mg/ml PGA and 0.25M NaCl is applied onto the molecular monolayer in the presence of 0.2M EDC and 50 mM NHS. That is, after proceeding a coupling reaction for an hour, a surface of the solid state element on which the molecular adhesive layer is formed is repeatedly washed with the triethanolamine(TEA) buffer solution(0.05M, pH 8.0).

(4) Formation of an interface sensing membrane-avidin coupling

In the immobilization of a bioelement in the three dimensional microstructured layer, avidin is selected as the bioelement for making coupling to the hydrophilic biopolymer. A TEA buffer solution containing 1 mg/ml avidin is applied onto the surface of the solid state element having up to the three dimensional microstructured layer formed thereon in the presence of 0.2M EDC and 50 mM NHS, and a coupling reaction is proceeded for an hour. Then, residual activated carboxyl functional groups are deactivated by 1M ethanolamine(pH 8.5), thereby completing fabrication of the interface sensing membrane.

(5) Biotinylated alkaline phosphatase(BAP) activity measurement

As the interface sensing membrane on the gold thin film contains avidin as the bioelement, the interface sensing membrane makes a specific coupling with the biotin. In order to quantify the biospecific interaction, an activity of the alkaline phosphatase having biotin derivates covalent bonded thereto is measured. 6 units of BAP is applied to the interface sensing membrane, which is placed in incubation for 30 min. Then, a surface of the interface sensing membrane is washed with a Tris buffer solution(10 mM, pH 8.0), to remove uncoupled BAP. An activity of BAP which has made a reaction with alkaline phosphatase immobilized in the interface sensing membrane using p-nitrophenol phosphate as a substrate is measured by spectrometry. An immobilized BAP activity is determined from an absorbed light measured at 410 nm after subjected to reaction at 25° C. for 10 min., and taking ε=18.8×10³M⁻¹cm⁻¹ as a molecular extinction coefficient, which are shown in TABLE 1, below. As shown in TABLE 1, the surface of the gold thin film without any treatment shows a great nonspecific binding. However, it is observed that the nonspecific binding onto the molecular adhesive layer is reduced significantly, and an immobilized BAP activity is enhanced significantly in the interface sensing membrane of the present invention.

TABLE 1
Comparison of BAP activities immobilized on
surfaces of different solid state element

| Surface | ΔAb_{S410}/10 min. | BAP unit(x10³) |
|---|---|---|
| Hydrophilic interface sensing | 0.083 | 0.44 |
| membrane | | |
| Cystamine surface | 0.001 | — |
| Gold thin film | 0.054 | 0.29 |

### EMBODIMENT 2

DNA interface, interaction between a DNA and a complementary DNA.

(1) Formation of a molecular adhesive layer on a gold thin film A process for forming the molecular adhesive layer on the gold thin film using a heterobifunctional reagent will be explained. A 3-mercaptopropionic acid(MPA) used as the heterobifunctional reagent has a thiol at one end and a carboxyl functional group at the other end. 1 mM of MPA solution is prepared using pure ethanol after deaeration by sonication. After 10 ml of the heterobifunctional reagent solution is filled in a glass scintillation vial, a specimen of the gold thin film is placed therein. Then, while being kept air tight, the vial is left standstill for more than 18 hours, to cause chemisorption reaction between the MPA solution and the gold thin film to form a molecular adhesive layer of a molecular monolayer. A surface of the molecular adhesive layer is washed with ethanol and water in succession, dried with nitrogen gas, and conserved in a nitrogen chamber.

(2) Formation of three dimensional microstructured layer on the molecular adhesive layer

The hydrophilic three dimensional microstructured layer can be formed on the molecular adhesive layer according to the following process. Poly-L-lysine(PL) is used as the hydrophilic polymer, and EDC and NHS are used as coupling reagent between the molecular adhesive layer, i.e., a molecular monolayer, and the hydrophilic polymer. A TEA buffer solution containing 0.2M EDC and 50 mM NHS is applied onto the molecular monolayer and left for one hour for reaction, to prepare an activated carboxyl surface. Then, after applying a TEA solution containing 1 mg/ml of PL to the surface, left for one hour for reaction, to form a three dimensional microstructured layer. Finally, remained activated carboxyl functional groups are deactivated by adding with 1M ethanolamine(pH 8.5), and washed with water adequately, and a surface is dried with an inert gas, such as nitrogen.

(3) Formation of an interface sensing membrane-cDNA coupling

0.5 mg/ml of specific cDNA of 1.0 kb amplified by PCR(Polymerase Chain Reaction) is applied to a surface of the interface sensing membrane, and dried. The immobilization by covalent adhesive of the cDNA with the PL which is a hydrophilic polymer is conducted as follows. The interface sensing membrane is left standstill in a humid chamber for 2 hours to rehydrate the interface sensing membrane, dried at 100° C. for 1 min., and washed with 0.1% SDS(Sodium Dodecyl Sulfate). After the washing, 0.05% succinic anhydride dissolved in 50% 1-methyl-2-pyrrolidinone and 50% boric acid is applied to the surface of the interface sensing membrane, thereby completing the interface sensing membrane with immobilized cDNA. When a complementary quantitative analysis of the DNA in the specimen is required, the interface sensing membrane is dipped into distilled water at 90° C. for 2 min. just before use, to denaturate the DNA into a single strand.

### EMBODIMENT 3

(1) Formation of a molecular adhesive layer on a silicon chip

A process for forming a molecular monolayer processed with a heterobifunctional reagent as the molecular adhesive layer on the silicon chip will be explained. A (3-aminopropyl) trimethoxysilane(APS) used as the heterobifunctional reagent has a silane functional group at one end and an amine functional group at the other end. A silicon wafer is cleaned and cut into 1×1 cm² size. A surface of the cut silicon chip is washed with anhydrous toluene containing 2% APS within a glove box kept air tight by an inert gas, such as argon, took out of the glove box, and dried by blowing nitrogen thereto. After the molecular monolayer is formed, the next step of formation of the hydrophilic three dimensional microstructured layer is started without delay.

(2) Formation of three dimensional micro structured layer on the molecular adhesive layer

The hydrophilic three dimensional microstructured layer can be formed on the molecular adhesive layer according to the following process. Poly-L-glutamate(PGA) is used as the hydrophilic polymer, and EDC and NHS are used as coupling reagent between the molecular adhesive layer and the hydrophilic polymer. A 0.05M of TEA buffer solution(pH 8.0) containing 1 mg/ml PGA and 0.25M NaCl is applied onto the molecular monolayer in the presence of 0.2M EDC and 50 mM NHS. In this instance, the NaCl used excessively reduces an electrostatic repulsion between carboxyl groups in the PGA. This coupling reagent is conducted for one hour. 1M ethanoldiamine(pH 8.5) is applied to substitute all activated carboxyl functional groups not participated in the reaction with amine terminated functional groups.

(3) Formation of antigen/antibody interface sensing membrane

An antigen can be immobilized at an interface sensing membrane according to the following process. A surface of the interface sensing membrane is dipped in dimethylformanide (DWF)/ethanol solution for one hour until 2 mM of m-Maleimidobenzoyl-N-hydroxydiimide ester(MBS), one of crosslinker, is present therein. After the reaction, the surface is washed with a PBS(Phosphate buffered saline solution) adequately, and applied of a monoclonal antibody to a hepatitis B surface antigen(HBsAg) at a concentration of 0.05 mg/ml. After leaving for one hour for reaction, the interface sensing membrane is dipped and stirred in PBS containing 0.1% Triton X-100 for 30 min, to clean the interface sensing membrane.

As explained, the interface sensing membrane in a bioelectronic device of the present invention can be used in multipurpose, allowing application to almost all kinds of surfaces of bioelements and solid state elements. The interface sensing membrane of the present invention can be employed as a surface of a transducer of bioelectronic device using an existing enzyme or antigen/antibody. And, if an immobilized DNA micron array is made available, the interface sensing membrane of the present invention is expected to be developed to a bioelectronic device having application to DNA base sequence analysis, genetic disease diagnosis, and detection of virus and bacteria behaviour. The antibody/antigen sensing membrane can be applied to medical diagnosis of infectious disease, and clinical and environmental multianalyte array for different measuring targets. The sensing membrane in which a bioelement making coupling with neuron is immobilized can be used in embodiment of a neurochip formed of a neuron network for use in information storage and processing.

It will be apparent to those skilled in the art that various modifications and variations can be made in the interface sensing membrane in a bioelectronic device and a method for forming the same of the present invention without departing from the spirit or scope of the invention. Thus, it is intended that the present invention cover the modifications and variations of this invention provided they come within the scope of the appended claims and their equivalents.

What is claimed is:

1. An interface sensing membrane in a bioelectronic device, comprising:
a molecular adhesive layer chemically adsorbed to a surface of a solid state element;
a three dimensional microstructured layer of a hydrophilic polymer connected to the molecular adhesive layer in a covalent bond; and,
a bioelement immobilized in the three dimensional microstructured layer by covalent adhesive.

2. An interface sensing membrane as claimed in claim 1, wherein the molecular adhesive layer is a molecular monolayer chemically adsorbed to the surface of the solid state element.

3. An interface sensing membrane as claimed in claim 1, wherein the solid state element is a gold thin film, a silver thin film, silicon or glass slide.

4. An interface sensing membrane as claimed in claim 1, wherein the three dimensional microstructur layer comprises of molecules of polypeptides.

5. An interface sensing membrane as claimed in claim 1 or 2, wherein the molecular adhesive layer comprises molecules each containing a functional group for making a chemical adsorption reaction with the surface of the solid state element and a functional group for making a covalent bond with the three dimensional microstructured layer.

6. An interface sensing membrane as claimed in claim 5, wherein the functional group for making a chemical adsorption reaction with the surface of the solid state element is sulfur, or silane.

7. An interface sensing membrane as claimed in claim 5, wherein the functional group for making a covalent bond with the three dimensional microstructured layer is carboxyl, aldehyde, amino, sulfhydryl, or hydrazide.

8. An interface sensing membrane as claimed in claim 1, wherein the three dimensional microstructured layer is formed of a material selected from a group of material including polyglutamic acid, polyaspartic acid, polylysine, and polycystein.

9. A method for forming the molecular adhesive layer of claim 1, comprising the step of causing the heterobifunctional reagent to make a reaction with the solid state element.

10. A method as claimed in claim 9, wherein the heterobifunctional reagent has the following chemical formula;
X-(CH,)n-Y, where, X denotes the functional group causing a chemical adsorption with the solid state element and Y denotes the functional group making a covalent bond with a hydrophilic polymer.

11. A method as claimed in claim 10, wherein the X is functional group containing sulfur, or silane, and the X is functional group containing carboxyl, aldehyde, amino, sulfhydryl, or hydrazide.

12. A method as claimed in claim 11, wherein the X is —SH, —S—SH, —SiCl₃, —Si(OCH₃)₃, or Si(OCH₂CH₃)₃.

13. A method for forming an interface sensing membrane in a bioelectronic device, comprising the steps of:
(1) applying a solution containing a heterobifunctional reagent to a surface of a solid state element, to form a molecular adhesive layer chemically adsorbed to the solid state element;
(2) causing a hydrophilic polymer to make a covalent bond with the molecular adhesive layer as a coupling reagent, to form a three dimensional microstructured layer thereon; and,
(3) connecting a bioelement to the three dimensional microstructured layer in a covalent bond.

14. A method as claimed in claim 13, wherein the molecular adhesive layer is a molecular monolayer chemically adsorbed to the solid state element.

15. A method as claimed in claims 13 or 14, wherein the step (1) includes the steps of,
(A) blowing an inert gas to the solid state element exposed to a heterobifunctional reagent, and
(B) drying the solid state element passed through the step (a) in a nitrogen chamber, to form a molecular adhesive layer in a form of a molecular monolayer.
